(19) United States
(12) Patent Application Publication
(10) Pub. No.: US 2025/0025596 A1
(43) Pub. Date: Jan. 23, 2025

(54) Adhesion Materials and Methods of Manufacture

(71) Applicant: BestHealth4U Unipessoal Lda, Braga (PT)

(72) Inventors: Sónia Carla Abrantes FERREIRA, Lisbon (PT); Nélson Duarte Mendes OLIVEIRA, Joane (PT)

(73) Assignee: BESTHEALTH4U UNIPESSOAL LDA, Braga (PT)

(21) Appl. No.: 18/903,342

(22) Filed: Oct. 1, 2024

Related U.S. Application Data
(63) Continuation-in-part of application No. 16/636,127, filed on Feb. 3, 2020.

(30) Foreign Application Priority Data
Nov. 6, 2017 (PT) 20171000073487

(51) Int. Cl. A61L 15/58; A61F 13/00; A61F 13/0246; C08K 5/092; C08K 5/1535; C09J 171/02

(52) U.S. Cl. CPC A61L 15/585; A61F 13/0253; C09J 171/02; A61F 2013/00604; A61F 2013/00659; C08K 5/092; C08K 5/1535; C09J 2471/00

(57) ABSTRACT

An adhesion material configured to be applied on a skin of a subject comprising a polymer molecular chain composition comprising polyethylene glycol 200 (PEG 200), polyethylene glycol 400 (PEG 400), citric acid, ascorbic acid, at least one of a sugar and a sugar alcohol, and at least one fatty acid.

### CROSS-REFERENCE TO RELATED APPLICATIONS

The present application claims is a continuation-in-part of U.S. patent application Ser. No. 16/636,127 filed by the present inventors on Feb. 3, 2020, which is a U.S. National Stage Application of PCT/EP2018/080359 filed on Nov. 6, 2018, which claims priority to Portugese Application Serial No. 20171000073487 filed on Nov. 6, 2017.

The aforementioned patent applications are hereby incorporated by reference in their entirety.

### BACKGROUND OF THE INVENTION

### Field of the Invention

The invention relates generally to adhesion materials and a method of their production. More particularly, the present invention relates to a new type of adhesive including the adhesion material that binds a pad, devices or other objects to the skin through hydrogen bonds.

### Brief Description of the Related Art

An adhesive is a product that can be applied to almost every type of medical device to bind the medical device to a skin of a person. For example, the adhesive can be applied to a simple wound dressing to enable the wound dressing to bind more tightly to the skin or to a more complex product, such as but not limited to, an ostomy bag that needs to be held in place on the skin.

In the market for medical adhesives there are several solutions known that comprise a mixture of one or more adhesion materials, such as acrylate, hydrocolloid or silicone. In recent years, there have been extensive advances in the adhesives industry which have produced new advances in the adhesion materials in a widening spectrum. For example, it is possible to produce variations of the adhesion material by adding different materials to the final composition and thereby enable properties of the adhesion materials to be tailored to meet specific application requirements, including biostability, sterilization effects, and mechanical properties.

The main types of adhesion materials used currently in the medical device area are cyanoacrylate, fibrin glue, GRF (Gelatin, resorcinol, formaldehyde), hydrogel and hydrocolloid and other adhesion materials with small market share or in research as the silicones, polyethylene glycol, albumin, chitosan, gelatin. These adhesion material compositions have known disadvantages, such as stripping (removing the adhesion material from the surface), mechanical injury due to tension, allergic contact resulting in dermatitis, folliculitis and skin maceration.

Beside the afore-mentioned disadvantages, a hydrocolloid dressing also presents the further disadvantages that the hydrocolloid dressing may dislodge from contact with the skin due to shearing or friction. The hydrocolloid dressing is also not recommended for use with infected wounds due to fluid discharge as the hydrocolloid dressing will also dislodge from the wound with heavy drainage. Finally, the odor when the hydrocolloid dressing is removed can be very strong and the removal may injure fragile skin.

### PRIOR ART

As was explained before, in this market it is possible to find several types of adhesion materials that use different strategies to stick to the skin, such as chemically, electrostatically or mechanically. The chemical adhesion materials comprise a large quantity of materials, such as but not limited to acrylic, styrene-isoprene-styrene block copolymers, high molecular weight polyisobutylenes loaded with glycols, fat, oil, glycerol esters or surfactants.

Silicone adhesives are also widely employed as skin friendly adhesives. In the patent document, EP 2853573 A1, for example, a diol was reacted with dicarboxylic acid through the polycondensation method, to develop an adhesion material which is friendly towards the skin.

According to the U.S. Pat. No. 5,308,887 A and international patent application No WO 2017/021448 A1 there are some pressure sensitive adhesives that are applied in strips, swirls, or waves. This type of adhesive refers to any releasable adhesive or releasable tenacious means. Suitable adhesion materials include, water-based pressure-sensitive adhesion materials, such as acrylate adhesives, natural rubbers, synthetic rubbers, vinyl acetates, silicones and polyurethanes. These adhesion materials can be based on emulsion or solvent-borne adhesives of natural or synthetic polyisoprene, styrene-butadiene, or polyacrylate, vinyl acetate copolymer.

Another type of adhesion material called hot melt adhesives is also known. According to the patent number EP 2756830 B1, a pressure-sensitive hot melt adhesive comprises substantially saturated block copolymer, tackifying resin, and polybutene. This hot melt composition exhibits adhesive stability when exposed to ultra-violet light and is stable when contacted with plasticized surfaces. These block copolymers include linear or radial co-polymer structures having the formula (A-B) wherein block A is a polyvinylarene block and block B is a poly(monoalkenyl) block. Block A includes polystyrene, polyalpha-methylstyrene and polyvinyltoluene. Block B can include conjugated diene elastomers such polybutadiene or polyisoprene or hydrogenated elastomers such as ethylene butylene or ethylene propylene or polyisobutylene.

Commercial examples of these types of block copolymers include Kraton™ elastomers from Shell Chemical Company, Vector™ elastomers from Dexco, Solprene™ from Enichem Elastomers and Stereon™ from Firestone Tire & Rubber Co.; hot melt adhesive based on olefin polymers and copolymers where in the olefin polymer is a terpolymer of ethylene and co-monomers, such as vinyl acetate, acrylic acid, methacrylic acid, ethyl acrylate, methyl acrylate, n-butyl acrylate vinyl silane or maleic anhydride. Commercial examples of these types of polymers include Ateva (polymers from AT plastics), Nucrel (polymers from DuPont), Escor (from Exxon Chemical).

Another type of adhesion material existing in the market is called a gecko solution. This gecko solution is a combination of microfibers and nanofibers that connect to the skin using a mechanical and electrostatic connection that results from the cumulative effect of van der Waals forces between the millions of setae on the feet pads in intimate contact with the surface the gecko is climbing on. The patent application US No 2014/0272272A1 uses an electrostatic dry adhesive device having a micro-structured element formed directly into a contact surface of an electrostatic adhesive. The micro-structure in the form of microwedges can be molded into surface of an electrostatic adhesive. This document refers to the use of at least one electrode that comprises a conductive mesh or fabric.

US Patent Application Publication No 20120288680 A1 uses a micro-featured and nano-featured surface, and a compliant surface having a hardness of about 60 Shore A or less. This type of adhesion composition needs flat surfaces for adhesion, such as polymer, metal or other.

The study “Adhesion and friction of an isolated gecko setal array: The effects of substrates and relative humidity”, by Y. Tian, showed that water affects the adhesion of the setal array by changing the Hamaker constant, and when the gecko encounters a surface with a higher surface energy or a more humid ambient environment, the gecko enjoys climbing more flexibly. Unfortunately, and according to the study “Surface free energy of the human skin and its critical surface tension of wetting in the skin/surfactant aqueous solution/air system,” by Krawczyk J, the skin surface is a low-energetic one. The critical surface tension of the skin wetting depends on the type of surfactant. In this respect, non-ionic surfactants seem to be the most appropriate.

Maleic anhydride has also been studied as an adhesion material. The researcher Hernández Sierra J F studied the application of this type of materials in “Bactericidal capacity of silver nanoparticles associated with Gantrez S-97 on Streptococcus mutans”. He concluded that the addition of Gantrez 2% to silver nanoparticles does not alter its antimicrobial effect. [Hernández, Sierra J F, Salas, López E K, et al. “Bactericidal capacity of silver nanoparticles associated with Gantrez S-97 on Streptococcus mutans.”; 2010 Winter; 35(2):183-5.] Anastasia Ripolin studied the application of this type of material in microneedle patches to be used in humans. She concluded that the use of such larger patches by patients can be successful, potentially opening up the possibility for a significant expansion of the size of the market for transdermal drug delivery. [Ripolin, Anastasia; James, Quinn; et al. “Successful application of large microneedle patches by human volunteers”. International Journal of Pharmaceutics, Vol. 521, Issues 1-2, 15 April 2017, 92-101.] E. Blanco Garcia has studied microspheres (Ms) based on zein (ZN) and Gantrez®AN119 (PVMMA) that were prepared by spray-drying and coated with a pH-sensitive polymer (Eudragit® FS30D) in intestinal inflammatory disorders. The authors concluded that ZN/PVMMA microspheres is a serious alternative for delivering the medicine to reduce the inflammatory activity at intestinal regions affected by inflammatory bowel diseases. [Garcia, E. Blanco; Espinar, F. J. Otero; “Development and characterization of anti-inflammatory activity of curcumin-loaded biodegradable microspheres with potential use in intestinal inflammatory disorders”. International Journal of Pharmaceutics, Volume 518, Issues 1-2, 25 Feb. 2017, 86-104]. This affinity has been studied and similar solutions patented by other authors, one of the studies showed the capability of the maleic anhydride to connect with the hydroxy or hydroxyl group.

The functionalization of polymers with this additive material is disclosed in the U.S. Pat. No. 6,451,919 B1 (1998). The patent teaches how the polyolefins can be functionalized with maleic anhydride and at least one high-boiling ester of the latter, and a process for their preparation in high yields, with high degrees of functionalization, without the production of by-products of cross-linking or degradation is disclosed.

In U.S. Pat. No. 8,940,132 are disclosed the interface modifiers, e.g., surface active agents, that may be used in functionalized polymers, preferably maleic anhydride grafted polyolefins, to improve the adhesion with cellulosic pulp fibers.

U.S. Pat. No. 8,940,278 focused on the efficacy enhancing agent that preferred copolymers of maleic anhydride or acid with another polymerizable ethylenically unsaturated monomer. The maleic anhydride was also used in the U.S. Pat. Nos. 8,906,406 and 8,907,153 for a mucoadhesive medicinal containing at least one active substance, in which a matrix comprises a mixture that comprises sodium carboxymethyl cellulose, polyvinyl alcohol and hydroxypropylmethyl cellulose. The adhesion material comprises at least one type of cellulose, derivatives, and for example maleic acid anhydride. As matrix-forming polymers which can be components of a mucoadhesive formulation, the following polymers were taken into consideration as raw materials: Polyvinyl alcohol (e.g. Mowiol®; cellulose derivatives, especially the types of Polyox 10, Polyox 80, Polyox 205, Polyox 301, Polyox 750 (made by the firm of Union Carbide); copolymers of methyl vinyl ether and maleic acid anhydride (Gantrez-Copolymers, especially the types of ES, MS, S, by the firm of ISP Global Technologies GmbH). According to the U.S. Pat. No. 8,940,132, it is preferably, the surface-active agent(s) presented in an amount greater than 2% by weight and less than 15% by weight of the entire composition of the composite, and more preferably in an amount less than or equal to 10% by weight.

Further patent applications are known which describe similar compositions. For example, International Patent Application No. WO 2017/075320 describes an antimicrobial adhesive made of silicone.

International patent application No. WO 2010/043346 teaches an aqueous solution of materials, or materials solubilized in conventional cosmetically or dermopharmaceutically acceptable solvents selected from the group consisting of ethanol, propanol, isopropanol, propylene glycol, glycerin, butylene glycol, or polyethylene glycol and mixtures thereof.

US patent application No. US 2013/0052236A1 teaches an adhesive based on mucoadhesive aspect comprises an alginate, phloroglucinol, maleic acid and an alkyl vinyl ether, as well as glycerol.

US patent application No. US 2007/0009582A1 teaches a plasticizer. This is only used to reduce the melting temperature. The adhesive is made only from hydrophobic block(s) consisting essentially of polymerized (meth)acrylic ester. The glycerol here is used as said aqueous solution and not to be used to create hydrogen bonds.

US Patent application No. US 2011/0105604 A1 discloses a cleansing and moisturizing composition. The composition comprises polyethylene glycol PEG (PEG)-30 diopolyhydroxystearate, citric acid, ascorbic acid, aloe barbadensis leaf juice, and lauric acid.

International patent application No. WO 2017/075320 A1 discloses an antimicrobial composition comprising PEG-8. WO 2017/075320 A1 further discloses an adhesive composition comprising a hydrophilic additive that may be selected from citric acid and its salts, glycerol, glycerol esters, monosaccharides, and disaccharides. The glycerol esters may be selected from unsaturated fatty acids including oleic acid.

European patent application No. EP 2789335 A1 discloses an adhesive agent layer comprising polyvalent alcohols, the polyvalent alcohols may be selected from polyethylene glycol, a pH-adjusting agent that may be selected from citric acid, and an antioxidant that may be selected from ascorbic acid.

U.S. Pat. No. 8,907,153 B2 discloses an adhesive peelable formulation comprising PEG-400 and oleic acid.

### SUMMARY OF THE INVENTION

An adhesion material is taught in this disclosure. The adhesion material is configured to be applied on a skin of a subject. The adhesion material comprises a polymer molecular chain composition (i.e., polymer chain with the chain having the following composition) comprising polyethylene glycol 200 (PEG 200), polyethylene glycol 400 (PEG 400), citric acid, ascorbic acid, at least one of a sugar and a sugar alcohol, and at least one fatty acid.

The adhesion material presented in this document is used in a novel type of adhesion material to secure a medical device to the skin of a human or an animal. This adhesion material was developed to overcome the main disadvantages of the current types of adhesion materials, namely the mechanical injury due to tension, allergic contact dermatitis, folliculitis and maceration. To overcome the disadvantages of the current adhesion materials, the adhesion material developed has the capability to bind to the water molecules that exist on top of the skin of the human or animal and not directly to the skin itself. To achieve this, the adhesion material uses hydrogen bonds. The binding with the hydrogen bonds reduces the number of skin cells that are removed on removal of the material in the prior art adhesion material, since the adhesion material taught in this document is not in permanent contact with the skin.

As was explained above, the adhesion material developed is based on the principle of hydrogen bonds. The hydrogen bonds are an attractive force between the hydrogen atom attached to an electronegative atom of one molecule and an electronegative atom of a different molecule. Usually the electronegative atom is oxygen, nitrogen, or fluorine, which has a partial negative charge. The hydrogen has a partial positive charge. This bond is not nearly as strong as normal covalent bonds within a molecule (about 1/10 as strong). However, this strength is enough to have important ramifications on the properties of water.

To achieve a good adhesion between the adhesion material of this document and the skin, the atoms of O···H need to be at distance of around 2.06 Å apart. At this distance, there exists both an electrostatic and covalent component of the bonding. If the distance between the two atoms increases or the amount of water is reduced, the adhesion strength of the adhesion material will be reduced to substantially zero, which will help to remove the medical device that is held to the skin by the adhesion material of this document. With this, the problems known in the art such as stripping, mechanical injury due to tension, allergic contact dermatitis, folliculitis, maceration are eliminated or at least substantially reduced.

In one aspect, the sugar is selected from the group of carbohydrates comprising sucrose, lactose, maltose, fructose, and combinations thereof, the sugar alcohol is selected from the alcohols comprising xylitol, sorbitol, and combination thereof, and the fatty acid is selected from at least one of lauric acid, oleic acid, and stearic acid.

In one aspect, the sugar alcohol is xylitol, and the fatty acid is stearic acid.

In one aspect, the adhesion material comprises 4% to 6% by weight of polyethylene glycol 400.

In one aspect, the adhesion material comprises lignin.

In one aspect, the adhesion material comprises 80% to 95% by weight of the polymer molecular chain composition and 5% to 20% by weight of lignin.

In one aspect, the adhesion material is a composition that comprises a product resulting from the reaction of polyethylene glycol (PEG), preferably from 200 to 8000 g/mol, with a sugar, such as sucrose, maltose, lactose, or fructose, and/or a sugar alcohol (also called polyhedric alcohol), such as xylitol (152 g/mol) or sorbitol, castor oil (150 g/mol), ascorbic acid (176 g/mol), citric acid (190 g/mol), lignin (1513 g/mol), oleic acid (284.47 g/mol) and lauric acid (200 g/mol).

It is known that PEG is a linear molecule in which the OH groups are localized at the end of the molecular chain. This localization will allow controlling the flexibility of the final material. The other materials due to their quantity of the hydroxy groups —OH will allow an increase in the hydrogen bonds in the adhesion material and thus between the adhesion material and the water in the skin. The lauric acid, stearic acid, or oleic acid, will provide water repellence. It was found out that PEG 200 in the mixture of the adhesion material results in a composition less toxic to humans than glycerol-containing composition.

To the previous composition is added one of the following compounds in manner to promote the necessary adhesion: polypropylene grafted with maleic anhydride or Polyethylene grafted with maleic anhydride or poly (ethylene-alt-maleic anhydride) or polystyrene-block-poly(ethylene-ran-butylene)-block-polystyrene-graft-maleic anhydride or polyisoprene-graft-maleic anhydride or poly(methyl vinyl ether-alt-maleic anhydride) or poly(isobutylene-alt-maleic anhydride) or poly(maleic anhydride-alt-1-octadecene) solution.

The materials presented above have on their structure a molecule that has affinity with water. This affinity is given mainly due to the maleic anhydride molecule. This molecule is reported by Arieny Rodrigues, in the study “Effect of compatibilization and reprocessing on the isothermal crystallization kinetics of polypropylene/wood flour composites”, in Polimeros, vol. 23, no. 3, Sio Carlos, 2013. The presence of a compatibilizer between two materials, such as a polymer and wood, decreases the interfacial tension, increasing the adhesion between the two materials and provides wettability, thus preventing agglomeration. In the market, there exist several materials grafted with maleic anhydride. It is possible to find materials with a concentration of this additive of between 1 to 10% in weight.

There are a large number of materials that are grafted with these additives and suitable ones of the materials can be selected by defining requirements according with the end application. To be more specific the material will be selected based on the level of erythema (redness of skin) caused by the material, the capability to create the adhesion on the skin at the temperature of the body (i.e., between 37° C. and 40° C.) and the capability to support a weight of 1 kg during 3 days without losing adhesiveness.

Another factor that will be considered to select the material is the manufacturing technique. According to European Chemicals Agency (ECHA, https://echa.europa.eu/substance-information/-/substanceinfo/100.003.247), maleic anhydride may cause irritation to the respiratory tract, eyes, and exposed skin. Maleic anhydride is also a skin and respiratory sensitizer.

Lignin is another material that can be used in the adhesion material, since lignin is an amorphous natural polymeric material that is based on a phenylpropane derivate, with large number of hydroxyl groups on their molecule chain, one of the most abundant materials and renewable resources on earth. The lignin-based adhesives have potential for engineering applications due to their environmental suitability, and economic and technical feasibility.

The manufacturing techniques that can be used to add the adhesion material to the medical device are the nano-spray, nano-printing and polymer brush. The polymer brush is a technique that is described by William J. Brittain in the work “A Structural Definition of Polymer Brushes”, Journal of Polymer Science: Part A: Polymer Chemistry, Vol. 45, 3505-3512 (2007). A polymer brush is an array of macromolecular chains which is attached or tethered to a surface and in sufficient proximity so that the unperturbed solution dimensions (in a good solvent) of the chains are altered. In the study, Brittain inferred that, to obtain a good alignment, it is necessary that the polymers have a low molecular weight of 14,500 g/mol.

A non-limiting example of the adhesive patch with the adhesion material will now be described. The adhesive patch has the following layers: a) an external layer made of a waterproof and breathable material (as for example ePTFE), silicone, thermoplastic polyurethane (TPU), polyether block amide (PEBA), or ethylene-vinyl acetate (EVA), with a thickness between 50 μm-500 μm); b) an intermediate layer made of plastic film, or a non-woven or woven fabric (non-limiting examples include polyamide, polyester, cotton, fibers of polypropylene, or polyethylene); and c) an inner layer of the adhesion material.

The inner layer is made of the adhesion material taught above and can interact with the water that exists in the skin. This adhesion material is composed by a mixture or a reaction result of polyethylene glycol and citric acid and ascorbic acid and glycerol as well as lauric acid and/or oleic acid with polypropylene grafted with maleic anhydride or polyethylene grafted with maleic anhydride or) or poly (ethylene-alt-maleic anhydride) or polystyrene-block-poly(ethylene-ran-butylene)-block-polystyrene-graft-maleic anhydride or polyisoprene-graft-maleic anhydride or poly(methyl vinyl ether-alt-maleic anhydride) or poly(isobutylene-alt-maleic anhydride) or poly(maleic anhydride-alt-1-octadecene) or poly(ethylene glycol)methyl ether methacrylate solution polyethylene glycol, with a polyol.

A method for manufacturing an adhesion material is taught in this disclosure. The method comprises drying polyethylene glycol 200, polyethylene glycol 400 and at least one of a sugar and a sugar alcohol, mixing the polyethylene glycol 400 and citric acid in a chemical reactor. The drying is conducted at temperatures between 60° C. and 100° C. and at least 1 hour to 10 hours prior to the mixing. The method further comprises blending the polyethylene glycol 200 and the sugar or the sugar alcohol in the chemical reactor, and blending at least one fatty acid, such as lauric acid, oleic acid, or stearic acid, in the chemical reactor. The method further comprises adding a base into the chemical reactor, so that the pH in the chemical reactor is between 5 and 7. Blending of ascorbic acid is subsequently conducted in the chemical reactor, thereby obtaining the adhesion material. As noted above, the adhesion material can be applied on a skin of a subject.

The term “blending” is intending to encompass both addition of material and mixing of the material.

The method for obtaining the adhesion material is based on the chemical processes of esterification and copolymerization. The esterification and the copolymerization occur substantially simultaneously in the chemical reactor.

In one aspect, the mixing, the blending, and the adding is conducted under at least one of vacuum or nitrogen, at temperatures between 100° C. and 200° C., at a mixing velocity between 1 and 2000 rpm, and for a duration between 1 hour to 48 hours.

In one aspect, the method is a closed batch method.

In one aspect, at least two of the mixing, the several blending, and the adding, are conducted substantially simultaneously.

In one aspect, the method further comprises mixing at least one of lignin and at least one of a polypropylene grafted with maleic anhydride, a polyethylene grafted with maleic anhydride, polystyrene-block-poly(ethylene-ran-butylene)-block-polystyrene-graft-maleic anhydride, polyisoprene-graft-maleic anhydride, poly(methyl vinyl ether-alt-maleic anhydride), poly(isobutylene-alt-maleic anhydride), poly(maleic anhydride-alt-1-octadecene) and a poly(ethylene glycol)methyl ether methacrylate solution to the adhesion material.

In one aspect, the mixing is conducted at temperatures between 23° C. and 130° C., at a mixing velocity between 1 and 1000 rpm, and for a duration between 1 min and 24 hours.

An adhesive patch is also disclosed. The adhesive patch comprises the adhesive material, an intermediate layer and an external layer, wherein the adhesive material being in contact with the skin, and the intermediate layer is made of a fabric, and the external layer is made of a waterproof and breathable material.

In one aspect, the intermediate layer is made of at least one of a non-woven and a woven fabric.

In one aspect, the external layer is at least one of expanded polytetrafluoroethylene (ePTFE), silicone, thermoplastic polyurethane (TPU), polyether block amide (PEBA), and Ethylene-vinyl acetate (EVA).

### BRIEF DESCRIPTION OF THE DRAWINGS

For easier understanding, the figures and tables representing preferential embodiments do not intend to limit the object of the present description.

FIG. 1 shows the molecular structure of polypropylene grafted with maleic anhydride;

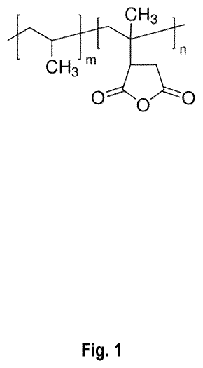

FIG. 2 shows the molecular structure of polyethylene grafted with maleic anhydride.

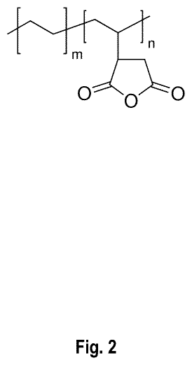

FIG. 3 shows the molecular structure of poly (ethylene-alt-maleic anhydride).

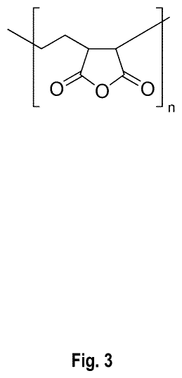

FIG. 4 shows the molecular structure of polystyrene-block-poly(ethylene-ran-butylene)-block-polystyrene-graft-maleic anhydride.

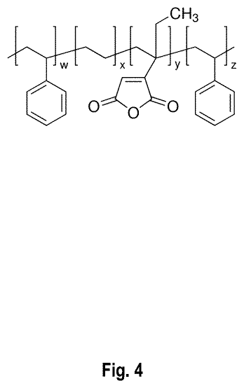

FIG. 5 shows the molecular structure of polyisoprene-graft-maleic anhydride.

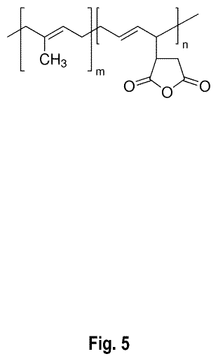

FIG. 6 shows the molecular structure of poly (methyl vinyl ether-alt-maleic anhydride).

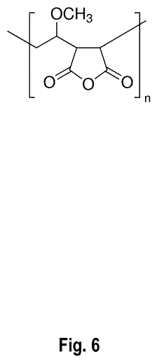

FIG. 7 shows the molecular structure of poly (isobutylene-alt-maleic anhydride).

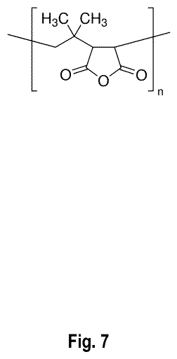

FIG. 8 shows the molecular structure of poly (maleic anhydride-alt-1-octadecene).

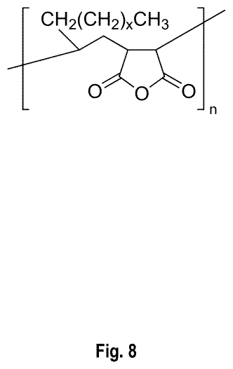

FIG. 9 is an example of how the materials can be placed.

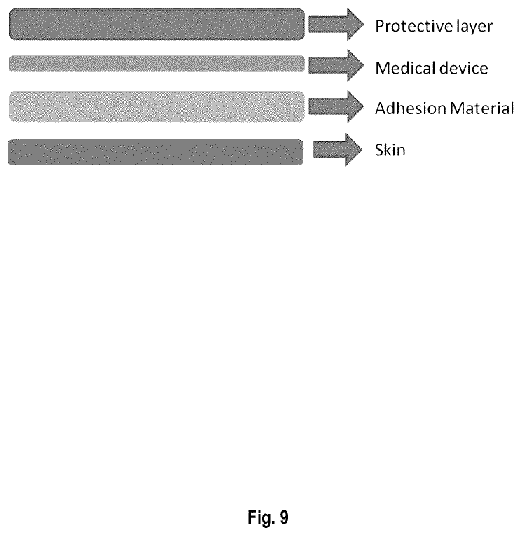

FIG. 10 shows the molecular structure resulted by the reaction between the different polyols.

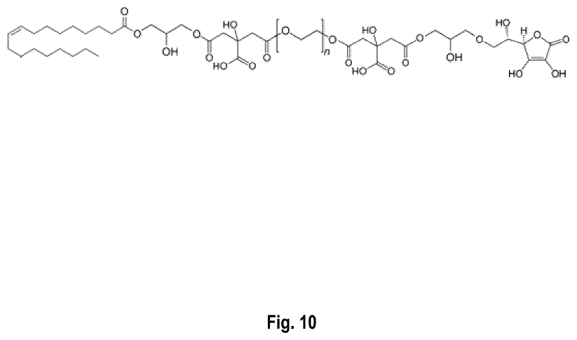

FIG. 11 shows the results of adhesion strength of the adhesive comparing with products already in the market.

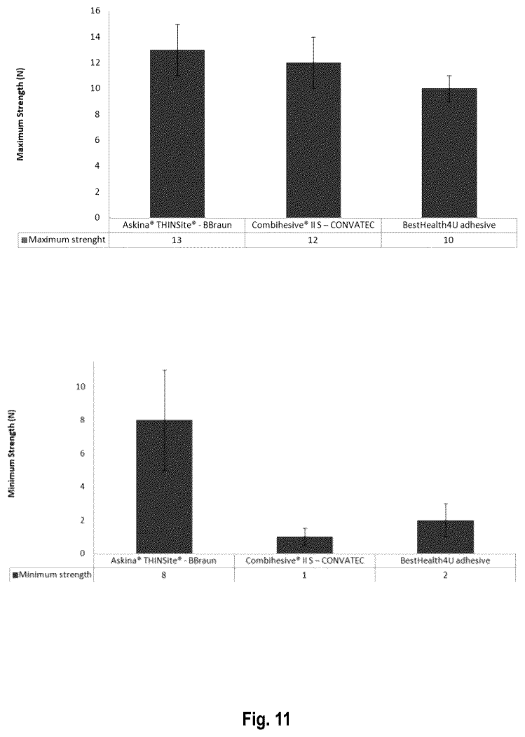

FIG. 12 shows the material attached to the skin.

FIG. 13 shows a chemical reactor and the obtention of the adhesion material.

FIG. 14 shows a flow chart describing a method for manufacturing the adhesion material.

FIG. 15 shows a structure of an adhesion patch comprising the adhesion material.

### DETAILED DESCRIPTION OF THE INVENTION

### Example 1

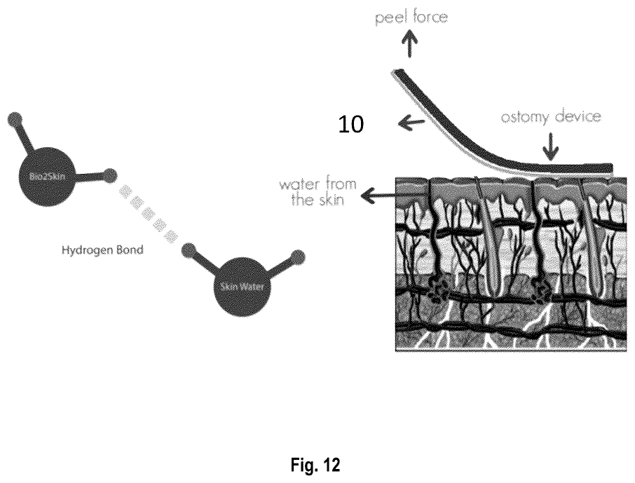

In this example the application of a composition will be illustrated. The composition used in the adhesion material 10 on the adhesive patch 50 as shown in the FIG. 12, is a mixture of
14% to 16% by weight of polyethylene glycol 400;
18% to 21% by weight of citric acid;
2% to 4% by weight of ascorbic acid;
4% to 6% in weight of polyethylene glycol 200;
5% to 7% in weight of xylitol; and
1% to 2% in weight of oleic acid.

The biopolymer resulting from the reaction of the different polyols is mixed, in this case is used between 44% and 56% in weight with the maleic anhydride polymer. The adhesion material 10 comprises 44% to 56% by weight of poly (methyl vinyl ether-alt-maleic anhydride). The materials to be used in the adhesion material 10 are mixed by hand or with the help of a mixing machine at temperatures between 23° C. and 90° C., and with a mixing velocity between 1 rpm and 1000 rpm. Poly (methyl vinyl ether-alt-maleic anhydride) was selected because this material presents the molecular length necessary to interact with the deep zone of the skin.

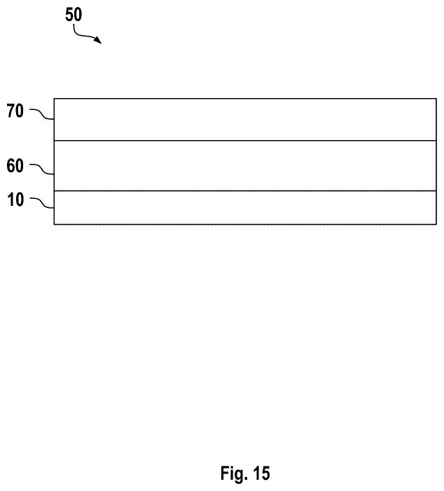

A plastic film or a fabric made of a non-woven or a woven fabric, such as but not limited to polyamide, polyester, cotton, fibers of polypropylene or polyethylene, is used as a substrate for the adhesive patch 50 and is illustrated on FIG. 15. This substrate is a layer of a material with the capability to protect the adhesion material and the skin 20 from atmosphere.

The adhesive patch 50 was obtained as follows. Firstly, an external layer 70 with a thickness between 50 μm and 500 μm of silicone was applied at room temperature to one of the faces of the non-woven or woven fabric. This silicone layer is applied at room temperature using the technique of knife-coating and/or spraying and/or printing. The protective layer was cured up to a maximum of three hours at temperatures between 23° C. to 100° C.

After this cure period the adhesion material 10 was added to the other one of the faces of the non-woven or woven fabric by knife-coating, nano-printing or nano-spray.

### Example 2

This example illustrates a way of producing an adhesion material 10 to be used in a hot and cold therapy adhesive patch 50. An adhesion material 10 of a butyl ester of methyl vinyl ether grafted with maleic anhydride was selected because this composition presents the molecular length necessary to interact with the deep zone of the skin 20 and has in its structure the maleic anhydride necessary to adhere to the skin 20.

The adhesion material 10 can be made through the following process. The polyethylene glycol 200, xylitol, and polyethylene glycol 400, are first dried, in an oven, at temperatures between 50° C. and 100° C., between 1 h and 24 h. Then they are added at same time or at predefined time point to a chemical reactor where they are mixed with citric acid, oleic acid and ascorbic acid. These acids are added together or at predefined time points. The materials are added into a chemical reactor, to perform the chemical reaction through the chemical processes named esterification and copolymerization. The whole chemical process can take between 1 h and 24 h, at temperatures between 100° C. and 180° C. The materials are mixed through the use of a mixer that rotates between 50 rotation per minute and 600 rotations per minute. The reaction can occur under vacuum or nitrogen, during whole time or predefined time points.

The composition to be used in the adhesion material 10 is a mixture of:
14% to 16% by weight of polyethylene glycol 400;
18% to 21% by weight of citric acid;
2% to 4% by weight of ascorbic acid;
4% to 6% in weight of polyethylene glycol 200;
5% to 7% in weight of xylitol; and
1% to 2% in weight of oleic acid.
   The polyol resulted from the reaction of the different polyols can also be used, in this case is used between 44% and 56% in weight. The adhesion material must have 44% to 56% in weight of a butyl ester of methyl vinyl ether grafted with maleic anhydride.

The materials were mixed for between 1 min to 24 hours at a temperature between 23° C. and 110° C., for example between 3° and 90° C. The materials were mixed between 1 min to 24 hours.

The adhesion material 10 is added to the face of the adhesive patch 50 that will be in contact with the skin 20 through knife-coating, nano-printing or nano-spray.

A protective layer of polyether block amide (PEBA) with a thickness of between 50 μm and 500 μm is added to the external face of the medical device. The protective layer will be used to protect the adhesion material from the atmosphere. The protective layer will be added to the adhesive patch by painting or thermoforming.

### Example 3

This example is a way of applying the adhesive material 10 to a wound dressing that uses a non-woven or woven fabric. A protective material is also used to protect the adhesion material 10 from the atmosphere, and the adhesion material 10 will enable adhesion to the skin 20. Poly (ethylene-alt-maleic anhydride) was used as the adhesion material 10 because this adhesion material 10 has the capability to interact with the water in the skin 20, as described above.

This adhesion material 10 was made through the following process. The polyethylene glycol 200, the xylitol, and the polyethylene glycol 400, are first dried, in oven at temperatures between 50° C. and 100° C., between 1 h and 24 h. Then they are added at same time or at predefined time point to a chemical reactor where they are mixed with citric acid, oleic acid and ascorbic acid. These acids are added together or at predefined time points. The materials are added into a chemical reactor, to perform the chemical reaction through the chemical processes named esterification and copolymerization. The whole chemical process can take between 1 h and 24 h, at temperatures between 100° C. and 180° C. The materials are mixed through the use of a mixer that rotates between 50 rotation per minute and 600 rotations per minute. The reaction can occur under vacuum or nitrogen, during whole time or predefined time points

The adhesion material 10 to be used in the adhesive is a mixture of
14% to 16% by weight of polyethylene glycol 400;
18% to 21% by weight of citric acid;
2% to 4% by weight of ascorbic acid;
4% to 6% in weight of polyethylene glycol 200;
5% to 7% In weight of xylitol;
1% to 2% in weight of oleic acid.

The polyol resulting from the reaction of the different polyols can also be used, in this case is used between 44% and 56% in weight. The adhesion material 10 must have 44% to 56% in weight of poly (ethylene glycol) methyl ether methacrylate solution.

To mix the different components of the adhesion material 10, temperatures between 23° C. and 110° C., and more preferably between 30-90° C., were used. The materials were mixed between 1 min to 24 hours. These components were mixed by hand or with help of a mixer with a mixing velocity between 1 rpm and 1000 rpm for between 1 min to 2 hours.

The adhesion material 10 was applied to one face of a non-woven fabric made of a polyamide or a layer of thermoplastic polyurethane (TPU) with thickness of between 50 μm and 500 μm.

### Testing

Tests regarding the adhesion capability of the adhesives were made, and these are shown in FIG. 12. These tests were made according with the standard ASTM D 3359-97 “tape test”. They compared the adhesion strength of the examples of this document invention with prior art materials, namely Combihesive IIS from Convatec and Askina “Thinsite” from BBraun. These tests evaluated the maximum and the minimum force needed to remove the adhesive. The figure shows that to remove the adhesive was necessary less force (ION) compared with the ones in the state-of-the-art (13N for Askina and 12 for Combihesive). Relative to the minimum force the adhesive developed needed more force (2N) compared to the Convatec (1N) but needed less force when compared to the product of BBraun (8N).

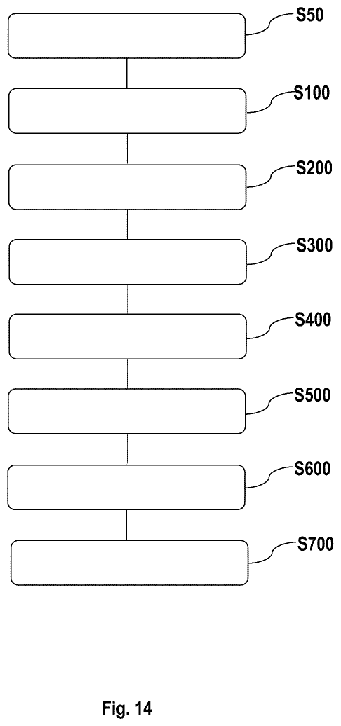

FIG. 14 is a block diagram representing the steps of the method for manufacturing the adhesion material 10. The method is based on two chemical processes, namely copolymerization and esterification. The copolymerization and the esterification are conducted substantially simultaneously. In a step S50, polyethylene glycol 200, polyethylene glycol 400 and at least one of a sugar and a sugar alcohol are dried. The drying of step S50 is conducted in a dry-type reactor. In one example, the drying is conducted in an oven, or any equipment configured to dry components. The drying of the step S50 is conducted at temperatures between 50° C. and 100° C. and at least 1 hour to 24 hours prior to a step S100 of mixing polyethylene glycol 400 and citric acid in the chemical reactor. The step S100 results in the formation of a polymer chain with a backbone structure of acid citric-PEG400-acid citric.

The step S100 of mixing is followed by a step S200 of blending polyethylene glycol 200 and at least one of a sugar or a sugar alcohol in the chemical 40. In one example, the sugar is sucrose, maltose, lactose or fructose, and/or the sugar alcohol is xylitol or sorbitol. In the example of xylitol as the sugar, the step S200 of blending results in the formation of a polymer chain with a backbone structure of PEG200-acid citric-PEG400-acid citric-xylitol.

The step S200 of blending is followed by a step S300 of blending at least one of a fatty acid, such as lauric acid, oleic acid, or stearic acid, in the chemical reactor 40. In one example, the step S300 comprises blending stearic acid in the chemical 40. In this example, the step S300 of blending results in the formation of a polymer chain with a backbone structure of stearic acid-PEG200-acid citric-PEG400-acid citric-xylitol.

The step S300 of blending is followed by adding in step S400 a base into the chemical reactor 40 so that the pH in the chemical reactor 40 is between 5 and 7. The step S400 of adding is followed by a step S500 of blending ascorbic acid in the chemical reactor 40. The step S500 of blending results in the formation of a polymer chain with a backbone structure of stearic acid-PEG 200-acid citric-PEG 400-acid citric-xylitol-ascorbic acid. Thus, the adhesion material 10 is obtained in a step S600.

In one example, the mixing of step S100, the blending of step S200, the blending of step S300, the adding of step S400 or the blending of step S500 is conducted under at least one of vacuum or nitrogen, at temperatures between 100° C. and 200° C., at a mixing velocity between 1 and 2000 rpm, and for a duration between 1 hour to 48 hours.

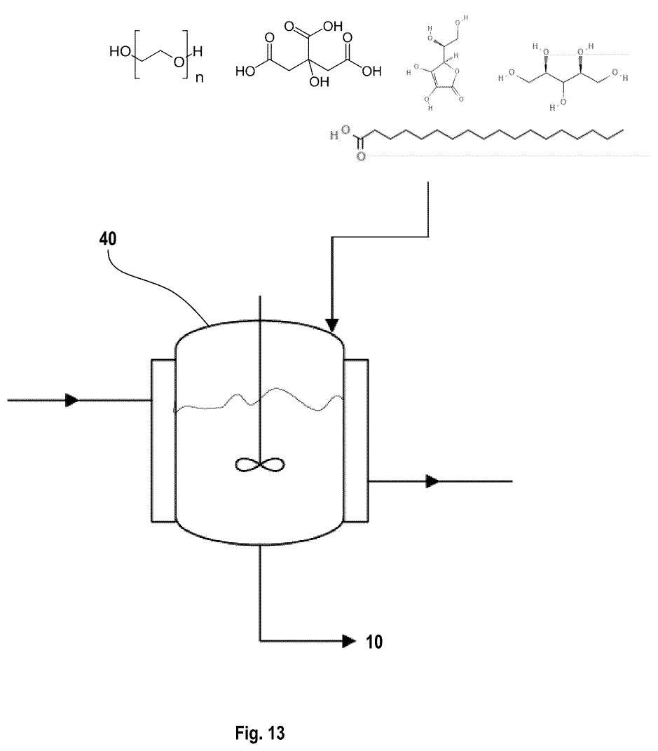

In one example and as illustrated in FIG. 13, the at least two of the mixing of step S100, the blending of step S200, the blending of step S300, the adding of step S400 or the blending of step S500 are conducted substantially simultaneously.

In one example, the method further comprises a step S700 of mixing at least one of lignin and at least one of a polypropylene grafted with maleic anhydride, a polyethylene grafted with maleic anhydride, polystyrene-block-poly(ethylene-ran-butylene)-block-polystyrene-graft-maleic anhydride, polyisoprene-graft-maleic anhydride, poly(methyl vinyl ether-alt-maleic anhydride), poly(isobutylene-alt-maleic anhydride), poly(maleic anhydride-alt-1-octadecene) and a poly(ethylene glycol)methyl ether methacrylate solution to the adhesion material 10, i.e., the obtained polymer molecular chain composition.

In one example, the step S700 of mixing is conducted at temperatures between 23° C. and 130° C., at a mixing velocity between 1 and 1000 rpm, and for a duration between 1 min and 24 hours.

In one further example, the step S700 of mixing comprises mixing 80% to 95% by weight of the polymer molecular chain composition and 5% to 20% by weight of the at least one of lignin and at least one of a polypropylene grafted with maleic anhydride, a polyethylene grafted with maleic anhydride, polystyrene-block-poly(ethylene-ran-butylene)-block-polystyrene-graft-maleic anhydride, polyisoprene-graft-maleic anhydride, poly(methyl vinyl ether-alt-maleic anhydride), poly(isobutylene-alt-maleic anhydride), poly(maleic anhydride-alt-1-octadecene) and a poly(ethylene glycol)methyl ether methacrylate solution to the adhesion material 10.

The materials with graft-maleic anhydride increase the adhesion of the adhesion material 10. The materials with graft-maleic anhydride are not biodegradable, so to improve the biodegradability of the adhesive material 10, the materials with graft-maleic anhydride in a range 5% to 20% by weight are added to the chemical reactor.

### Method for Manufacturing the Adhesion Material

| Materials used: | Equipment |
|---|---|
| PEG400 | Chemical Reactor |
| PEG200 | Vacuum pump |
| Ascorbic Acid | Temperature control unit |
| Citric Acid anhydrous | |
| Stearic acid | |
| Xylitol | |
| Potassium Bicarbonate | |

The drying step S50 was conducted 4 hours prior to the mixing and blending steps. PEG 400, PEG 200 and xylitol were dried in a standard oven at 75° C. In a dry chemical reactor were added PEG 200, and PEG400. The components inside the chemical reactor 40 were heated until the temperature of 90° C. is reached.

Citric acid was then added in the chemical reactor 40. The components inside the vessel of the chemical reactor 40 were heated until the temperature of 150° C. is reached. Vacuum was initiated when the temperature reached 145° C. in the chemical reactor 40. The vacuum pressure was 0.1 bar. The vacuum pump was turned off after 5 h of mixing and the xylitol was added into the chemical reactor 40.

After 10 min of mixing all the components, the vacuum pump was turned on again. After 4 h of mixing, the vacuum pump was turned off and stearic acid was added. The chemical reactor 40 was heated to the temperature of 165° C. after the addition of the stearic acid. The vacuum pump was turned-on after 10 min of mixing all the components.

The temperature in the chemical reactor 40 was subsequently reduced to 135° C. after 8 h of mixing. Potassium bicarbonate was added in small quantities into the chemical reactor 40. The temperature in the chemical reactor 40 was reduced to 130° C. after 3 h of mixing, and the vacuum pump was turned off.

Ascorbic acid was added into the chemical reactor 40. The vacuum pump was turned on after 10 min of mixing all the components. The vacuum pump was then turned turnoff after 1 hour of mixing. The adhesion material 10 was then removed from the chemical reactor 40 and stored into the oven during 24 h at 75° C. Then, the adhesive material 10 was stored in a dry place and protected from sun.

In one example, the mixing velocities of the method are 400 rpm.

FIG. 15 shows an adhesive patch 50 with the adhesive material 10 made according to the protocol set out above. The adhesive patch 50 comprises the adhesive material 10, an intermediate layer 60 and an external layer 70. The adhesive material 10 is in contact with the skin 20 with the intermediate layer 60. The intermediate layer 60 is in contact with the adhesive material 10 and with the external layer 70. In other words, the intermediate layer 60 is sandwiched between the adhesive material 10 and the external layer 70.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described to explain the principles of the invention and its practical application to enable one skilled in the art to utilize the invention in various embodiments as are suited to the particular use contemplated. It is intended that the scope of the invention be defined by the claims appended hereto and their equivalents. The entirety of each of the aforementioned documents is incorporated by reference herein.

### REFERENCE NUMERALS

10 adhesion material
20 skin
30 subject
40 chemical reactor
50 adhesive patch
60 intermediate layer
70 external layer
S50 drying
S100 mixing
S200 blending
S300 blending
S400 adding
S500 blending
S500 obtaining
S600 obtaining
S700 mixing

What is claimed is:

1. An adhesion material configured to be applied on a skin of a subject, the adhesion material comprising a polymer molecular chain composition comprising
polyethylene glycol 200 (PEG 200);
polyethylene glycol 400 (PEG 400);
citric acid;
ascorbic acid;
at least one of a sugar and a sugar alcohol; and
at least one fatty acid.

2. The adhesion material according to claim 1, wherein at least one of
the sugar is selected from the group of carbohydrates comprising sucrose, lactose, maltose, fructose, and combinations thereof;
the sugar alcohol is selected from the alcohols comprising xylitol, sorbitol, and combination thereof; and
the fatty acid is selected from at least one of lauric acid, oleic acid, and stearic acid.

3. The adhesion material according to claim 1, wherein
the sugar alcohol is xylitol; and
the fatty acid is stearic acid.

4. The adhesion material according to claim 1, comprising
4% to 6% by weight of polyethylene glycol 400.

5. The adhesion material according to claim 1, further comprising lignin.

6. The adhesion material according to claim 1, comprising 80% to 95% by weight of the polymer molecular chain composition and 5% to 20% by weight of lignin.

7. The adhesion material according to claim 1, further comprising at least one of a polypropylene grafted with maleic anhydride, a polyethylene grafted with maleic anhydride, polystyrene-block-poly(ethylene-ran-butylene)-block-polystyrene-graft-maleic anhydride, polyisoprene-graft-maleic anhydride, poly(methyl vinyl ether-alt-maleic anhydride), poly(isobutylene-alt-maleic anhydride), poly(maleic anhydride-alt-1-octadecene) and a poly(ethylene glycol)methyl ether methacrylate solution.

8. The adhesion material according to claim 1, further comprising poly (ethylene-alt-maleic anhydride).

9. The adhesion material according to claim 1, comprising
80% to 95% by weight of the polymer molecular chain composition; and
5% to 20% by weight of at least one of a polypropylene grafted with maleic anhydride, a polyethylene grafted with maleic anhydride, polystyrene-block-poly(ethylene-ran-butylene)-block-polystyrene-graft-maleic anhydride, polyisoprene-graft-maleic anhydride, poly(methyl vinyl ether-alt-maleic anhydride), poly(isobutylene-alt-maleic anhydride), poly(maleic anhydride-alt-1-octadecene) and a poly(ethylene glycol)methyl ether methacrylate solution.

10. A method for manufacturing an adhesion material, the method comprising
drying polyethylene glycol 200, polyethylene glycol 400 and at least one of a sugar and a sugar alcohol;
mixing the polyethylene glycol 400 and citric acid in a chemical reactor;
wherein the drying is conducted at temperatures between 60° C. and 100° C. and at least 1 hour to 24 hours prior to the mixing;
blending the polyethylene glycol 200 and the at least one of a sugar or a sugar alcohol in the chemical reactor;
blending at least one fatty acid in the chemical reactor;
adding a base into the chemical reactor so that the pH in the chemical reactor is between 5 and 7; and
blending ascorbic acid in the chemical reactor (40);
thereby obtaining the adhesion material, the adhesion material being configured to be applied on a skin of a subject.

11. The method of claim 10, wherein
the blending comprises blending at least one of lauric acid, oleic acid, and stearic acid, in the chemical reactor; and
at least one of the mixing, the blending, the blending, the adding, or the blending, is conducted
under at least one of vacuum or nitrogen;
at temperatures between 100° C. and 200° C.;
at a mixing velocity between 1 and 2000 rpm; and
for a duration between 1 hour to 48 hours.

12. The method according to claim 10, wherein the method is a closed batch method.

13. The method according to claim 10, wherein at least two of the mixing, the blending, the blending, the adding and the blending are conducted substantially simultaneously.

14. The method according to claim 10, further comprising
mixing at least one of lignin and at least one of a polypropylene grafted with maleic anhydride, a polyethylene grafted with maleic anhydride, polystyrene-block-poly(ethylene-ran-butylene)-block-polystyrene-graft-maleic anhydride, polyisoprene-graft-maleic anhydride, poly(methyl vinyl ether-alt-maleic anhydride), poly(isobutylene-alt-maleic anhydride), poly(maleic anhydride-alt-1-octadecene) and a poly(ethylene glycol)methyl ether methacrylate solution to the adhesion material.

15. The method of claim 14, wherein the mixing is conducted
at temperatures between 23° C. and 130° C.;
at a mixing velocity between 1 and 1000 rpm; and
for a duration between 1 min and 24 hours.

16. The adhesive material of claim 1 for use in a medical device, an adhesive patch, or kinesiology bands.

17. An adhesive patch comprising the adhesive material of claim 1, an intermediate layer and an external layer, wherein
the adhesive material being in contact with the skin, and at least one of
the intermediate layer is made of a fabric; and
the external layer is made of a waterproof and breathable material.

18. The adhesive patch of claim 17, wherein the intermediate layer (60) is made of at least one of a non-woven and a woven fabric.

19. The adhesive patch of claim 17, wherein the external layer is at least one of expanded polytetrafluoroethylene (ePTFE), silicone, thermoplastic polyurethane (TPU), polyether block amide (PEBA), and ethylene-vinyl acetate (EVA).
